(19) United States
(12) Patent Application Publication
(10) Pub. No.: US 2008/0139483 A1
(43) Pub. Date: Jun. 12, 2008

(54) DRUG COMBINATION FOR HYPERTENSIVE DISORDERS

(76) Inventor: Richard J. Gorczynski, Westminster, CO (US)

Correspondence Address: HARNESS, DICKEY, & PIERCE, P.L.C, 7700 Bonhomme, Suite 400, ST. LOUIS, MO, 63105, US

(21) Appl. No.: 11/953,937

(22) Filed: Dec. 11, 2007

Related U.S. Application Data
(60) Provisional application No. 60/869,661, filed on Dec. 12, 2006.

(51) Int. Cl. A61K 38/04; A61K 31/513; A61K 31/165

(52) U.S. Cl. 514/19; 514/269; 514/616

(57) ABSTRACT

Therapeutic combinations and pharmaceutical compositions are provided comprising ambrisentan and an inhibitor of renin activity or release. Such combinations and compositions are useful to treat hypertensive disorders, particularly pulmonary hypertension conditions such as pulmonary arterial hypertension, to prevent cardiovascular adverse events related thereto, to enhance pulmonary function and to extend time to clinical worsening in a subject having a pulmonary hypertension condition.

This application claims the benefit of U.S. provisional application Ser. No. 60/869,661, filed Dec. 12, 2006, incorporated in its entirety herein by reference.

### FIELD OF THE INVENTION

The present invention relates to therapeutic combinations, compositions, and methods useful for treating hypertensive disorders and preventing cardiovascular adverse events related thereto.

### BACKGROUND OF THE INVENTION

Pulmonary arterial hypertension (PAH) is a highly debilitating disease characterized by severe constriction of blood vessels in the lungs leading to excessively high pulmonary arterial pressures. These high pressures and a high pulmonary vascular resistance associated therewith make it difficult for the heart to pump blood through the lungs to be oxygenated. Patients with PAH suffer from extreme shortness of breath as the heart struggles to pump against these high pressures. Without treatment such patients can ultimately die of heart failure. PAH can occur with no known underlying cause (“primary PAH”), or it can occur secondarily to diseases such as scleroderma, connective tissue disease, congenital heart defects, cirrhosis of the liver and HIV infection.

Ambrisentan is an endothelin-A (ET_{A}) selective receptor antagonist which has been proposed for treatment of PAH and other pulmonary hypertension conditions. Endothelin is a small peptide hormone that is believed to play a critical role in control of blood flow and cell growth. Elevated endothelin blood levels are associated with several cardiovascular disease conditions, including not only PAH but also chronic renal disease, coronary artery disease, hypertension and chronic heart failure. Endothelin is a potent vasoconstrictor, triggering contraction through endothelin-receptor mediated signaling pathways.

Myogen, Inc. News Release, Dec. 4, 2003 (http://www.prnewswire.com/cgi-bin/stories.pl?ACCT=104&STORY=/www/story/12-04-2003/0002069898&EDATE=) announced completion of a Phase II trial of ambrisentan in PAH and initiation of Phase III trials. The release stated that the Phase III trials would evaluate 2.5 mg, 5.0 mg and 10.0 mg oral dosages of ambrisentan administered once a day, and would have as a primary efficacy endpoint exercise capacity, which measures the change from baseline in 6-minute walk distance (6MWD) compared to placebo, and secondary endpoints including Borg dyspnea index (BDI), WHO functional class and a quality of life assessment.

Myogen, Inc. News Release, Jan. 8, 2004 (http://investor.myogen.com/phoenix.zhtml?c=135160&p=irol-newsArticle&ID=759080&highlight=) announced patient enrollment in phase III clinical trials of ambrisentan for treatment of PAH. According to the news release, phase II trials had demonstrated a statistically significant and clinically meaningful increase in the primary efficacy endpoint (exercise capacity measured by 6MWD) in all four ambrisentan dose groups tested.

Myogen, Inc. News Release, Feb. 16, 2004 (http://investor.myogen.com/phoenix.zhtml?c=1135160&p=irol-newsArticle&ID=759478&highlight=) announced upcoming presentation of detailed results of the phase II study of ambrisentan in PAH, at the American Thoracic Society (ATS) 2004 International Conference. (Rubin (2004) “Ambrisentan Improves Exercise Capacity and Clinical Measures in Pulmonary Arterial Hypertension”, ATS May 21-26, 2004.

Myogen, Inc. News Release, May 24, 2004 (http://investor.myogen.com/phoenix.zhtml?c—135160&p=irol-newsArticle&ID=759469&highlight=) reported improvements in 6MWD, BDI and WHO functional classification seen in the Phase II study. Additionally, the news release mentioned suitability of ambrisentan for once-a-day dosing.

Myogen, Inc. News Release, Feb. 10, 2005 (http://investor.myogen.com/phoenix.zhtml?c=135160&p=irol-newsArticle&ID=759971&highlight=) announced that two abstracts describing effects of ambrisentan in patients with PAH were selected for presentation at ATS 2005 in San Diego. (Galié (2005) “Ambrisentan Long-Term Safety and Efficacy in Pulmonary Arterial Hypertension 1-Year Follow-Up”, ATS May 23, 2005; Frost (2005) “Ambrisentan Improves 6MWD Comparably for WHO Class II and III PAH Patients,” ATS May 22, 2005.) It was stated that one-year data demonstrated that ambrisentan produced a significant and durable benefit on exercise capacity and other clinical measures of PAH and that WHO Class II and III PAH patients have significant and comparable improvement in exercise capacity, suggesting that the effects of ambrisentan are not limited by the “ceiling effect” in patients with less severe PAH symptoms.

Myogen, Inc. News Release, May 19, 2005 (http://investor.myogen.com/phoenix.zhtml?c=135160&p=irol-newsArticle&ID=759658&highlight=) reported initiation of a clinical trial to evaluate ambrisentan in patients with PAH who have previously discontinuted bosentan or sitaxsentan therapy due to liver function test (LFT) abnormalities, specifically elevated serum aminotransferase concentrations.

Myogen, Inc. News Release, May 23, 2005 (http://investor.myogen.com/phoenix.zhtml?c=135160&p=irol-newsArticle&ID=759656&highlight=) reported further data presented by Galié (2005) ATS 2005, cited above, which were stated to show improvements in a 6-minute walking test (6MWT) accompanied with improved levels of dyspnea (breathlessness) for WHO Class II and III patients. The release reported a one-year survival rate of 92% for patients with idiopathic PAH as compared to an NIH registry predicted survival of 74%.

Myogen, Inc. News Release, Jul. 21, 2005 (http://investor.myogen.com/phoenix.zhtml?c=135160&p=irol-newsArticle&ID=759650&highlight=) announced completion of enrollment of 187 patients in ARIES-2, one of the two Phase III clinical trials of ambrisentan in patients with PAH. The news release reported that ARIES-1 evaluates doses of 5.0 mg and 10.0 mg of ambrisentan administered orally once daily, while ARIES-2 provides 2.5 mg and 5.0 mg dosages. The release stated that the results of the Phase II clinical trial of ambrisentan in patients with PAH demonstrated significant improvements in 6MWD, BDI and WHO functional class, durable efficacy with long-term use and a possible survival benefit, comparable efficacy in WHO Functional Class 2 and Class 3 patients, selectivity for the endothelin type-A receptor, dose flexibility, true once-daily dosing, no drug interactions (no p450 induction or inhibition), and low incidence and severity of potential liver toxicity that does not appear to be dose related.

Myogen, Inc. News Release, Nov. 10, 2005 (http://investor.myogen.com/phoenix.zhtml?c=135160&p=irol-newsArticle&ID=781654&highlight=) announced the expectation that ARIES-2 results would be reported in December of that year.

Rubin et al. (2005) Future Cardiol. 1(4):1-8 reported improvement of the mean 6MWD for all patients after 12 weeks of ambrisentan treatment, with a mean increase from baseline of 36 meters. The authors reported that similar improvements in 6MWD were observed for patients with WHO Functional Class II and III symptoms, indicating that the effects of ambrisentan may not be limited by a “ceiling effect” in less advanced PAH patients, as has been reported for sitaxsentan. Additionally, the authors reported that clinically meaningful improvements were also seen in BDI and WHO functional class.

Galieé et al. (2005) J. Am. Coll. Cardiol. 46(3):529-535 reported results of a randomized dose-ranging study examining efficacy and safety of ambrisentan in patients with PAH. The authors reported an increase in exercise capacity in patients with idiopathic PAH as well as in patients with PAH due to other etiologies and for patients in WHO Functional Class II as well as those in WHO Functional Class III.

Renin is an aspartyl protease secreted by the kidneys. Its primary substrate is angiotensinogen, which is secreted by hepatocytes. Renin cleaves angiotensinogen forming the decapeptide angiotensin I. Angiotensin I is then further cleaved in the lungs to release the octapeptide, angiotensin II. Angiotensin II increases blood pressure both directly by arterial vasoconstriction and indirectly by promoting the removal of aldosterone, a sodium-ion retaining hormone, from the adrenal glands, thus increasing extracellular fluid volume. Renin inhibitors can affect both the activity and release of renin from the kidney. By either affecting the activity or release of renin, less angiotensin I is formed from the cleavage of angiotensinogen. This results in less angiotensin II production and a reduction in blood pressure.

International Patent Publication No. WO 02/40007 of Novartis proposes combinations of the renin inhibitor aliskiren and any of a large number of therapeutic agents of many different classes for use in treating cardiovascular diseases such as hypertension and atherosclerosis. One such class mentioned is endothelin antagonists.

International Patent Publication No. WO 03/099767 of Novartis proposes certain amide derivatives as inhibitors of the enzymatic activity of renin, and combinations of such amide derivatives with any of a large number of therapeutic agents of many different classes for use in treating various disorders such as hypertension and congestive heart failure. One such class mentioned is endothelin antagonists.

International Patent Publication No. WO 04/100871 of Pharmacia proposes inter alia therapeutic combinations and compositions comprising an aldosterone receptor antagonist, a renin inhibitor and a third drug, which can be any of a large number of drugs of different classes. One such class mentioned is endothelin antagonists.

PAH afflicts approximately 200,000 patients worldwide. Improved drug therapies to treat hypertensive disorders such as PAH are needed in the art. Further, methods to prevent cardiovascular adverse events in hypertensive patients, especially patients with PAH, would be highly desirable.

### SUMMARY OF THE INVENTION

There is now provided a therapeutic combination comprising ambrisentan and an inhibitor of renin activity or release.

Such a combination can optionally take the form of a pharmaceutical composition comprising ambrisentan, the inhibitor of renin activity or release, and at least one pharmaceutically acceptable excipient.

There is further provided a method for treating a hypertensive disorder, for example PAH, in a subject. The method comprises administering to the subject in combination therapy ambrisentan and an inhibitor of renin activity or release in antihypertensive effective absolute and relative amounts.

There is still further provided a method for preventing one or more cardiovascular adverse events in a subject having a pulmonary hypertension condition, for example PAH, comprising administering to the subject in combination therapy ambrisentan and an inhibitor of renin activity or release.

There is still further provided a method for improving pulmonary function in a subject having a pulmonary hypertension condition, for example PAH, comprising administering to the subject in combination therapy ambrisentan and an inhibitor of renin activity or release.

There is still further provided a method for extending time to clinical worsening in a subject having a pulmonary hypertension condition, for example PAH, comprising administering to the subject in combination therapy ambrisentan and an inhibitor of renin activity or release.

Other embodiments, including particular aspects of the embodiments summarized above, will be evident from the detailed description that follows.

### DETAILED DESCRIPTION

In various aspects of the invention, therapeutic combinations and compositions comprising ambrisentan and an inhibitor of renin activity or release, and methods of use of such combinations and compositions, are provided.

As used herein, the term “renin inhibitor” means an inhibitor of enzymatic activity of renin. Suitable renin inhibitors include without limitation aliskiren, ciprokiren, ditekiren, enalkiren, remikiren, terlakiren and zankiren. Derivatives of these compounds, including salts, esters, prodrugs, metabolites, enantiomers, racemates and tautomers thereof having renin inhibitory properties are also suitable for use in the present invention, as are combinations of renin inhibitors and/or derivatives thereof. Inhibitors of renin release, and derivatives thereof, can likewise be used in the present combinations, compositions and methods.

The term “subject” refers to a warm-blooded animal, generally a mammal such as, for example, a primate, including a human. In one embodiment the subject is a human, for example a patient having hypertension, more particularly a pulmonary hypertension condition, for example PAH. Typically but not necessarily, PAH is diagnosed clinically in such a patient.

In one embodiment, the ambrisentan and the inhibitor of renin activity or release are present in the combination or composition, or used according to a method of the invention, in antihypertensive effective absolute and relative amounts. The term “absolute and relative amounts” as used herein refers to a first amount of ambrisentan and a second amount of an inhibitor of renin activity or release wherein the first amount and second amount together constitute an antihypertensive effective amount, for example an amount effective to lower pulmonary arterial blood pressure, whether or not the first amount or second amount alone would be effective.

In a particular aspect, the ambrisentan and the inhibitor of renin activity or release are present in absolute and relative amounts effective to lower pulmonary arterial blood pressure, particularly in a subject having PAH. Illustratively, the ambrisentan and the inhibitor of renin activity or release are present in absolute and relative amounts effective to lower pulmonary arterial blood pressure by at least about 3 mmHg, for example by at least about 5 mmHg.

Optionally one or more additional therapeutic agents can be present in the combination or composition, or administered in addition to the ambrisentan and the inhibitor of renin activity or release. Examples of such additional agents include prostanoids such as beraprost, cicaprost, epoprostenol, iloprost, NS-304, PGE₁, prostacyclin or treprostinil; phosphodiesterase inhibitors, especially phosphodiesterase type 5 (PDE5) inhibitors such as sildenafil, tadalafil and vardenafil; calcium channel blockers including arylalkylamines (e.g., bepridil, clentiazem, diltiazem, fendiline, gallopamil, mibefradil, prenylamine, semotiadil, terodiline and verapamil), dihydropyridine derivatives (e.g., amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine and NZ 105), piperazine derivatives (e.g., cinnarizine, dotarizine, flunarizine, lidoflazine and lomerazine) and unclassified calcium channel blockers (e.g., bencyclane, etafenone, fantofarone, monatepil and perhexyline); diuretics including organomercurials (e.g., chlormerodrin, meralluride, mercaptomerin sodium, mercumatilin sodium, mercurous chloride and mersalyl), purines (e.g., pamabrom, protheobromine and theobromine), steroids (e.g., canrenone, eplerenone, oleandrin and spironolactone), sulfonamide derivatives (e.g., acetazolamide, ambuside, azosemide, bumetanide, butazolamide, chloraminophenamide, clofenamide, clopamide, clorexolone, disulfamide, ethoxzolamide, furosemide, mefruside, methazolamide, piretanide, torsemide, tripamide and xipamide), thiazides and analogs (e.g., althiazide, bendroflumethiazide, benzthiazide, benzylhydrochlorothiazide, buthiazide, chlorothiazide, chlorthalidone, cyclopenthiazide, cyclothiazide, ethiazide, fenquizone, hydrochlorothiazide, hydroflumethiazide, indapamide, methyclothiazide, metolazone, paraflutizide, polythiazide, quinethazone, teclothiazide and trichlormethiazide), uracils (e.g., aminometradine) and unclassified diuretics (e.g., amiloride, Biogen BG 9719, chlorazanil, ethacrynic acid, etozolin, isosorbide, Kiowa Hakko KW 3902, mannitol, muzolimine, perhexyline, Sanofi-Aventis SR 121463, ticrynafen, triamterene and urea); and anticoagulants such as acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, pentosan polysulfate, phenindione, phenprocoumon, phosvitin, picotamide, tioclomarol and warfarin.

Any dosage of ambrisentan which, together with the inhibitor of renin activity or release, provides a beneficial effect without unacceptable adverse side-effects in a subject can be present in the combination or composition, or used according to a method of the invention. While in one embodiment the ambrisentan is administered orally, the invention is not limited to any route of administration, so long as the route selected results in effective delivery of the drug to provide a beneficial effect. Thus administration of the ambrisentan can illustratively be parenteral (e.g., intravenous, intraperitoneal, subcutaneous or intradermal), transdermal, transmucosal (e.g., buccal, sublingual or intranasal), intraocular, or rectal. Most conveniently for the majority of patients, however, the ambrisentan is administered orally, i.e., per os (p.o.). Any suitable orally deliverable dosage form can be used for the ambrisentan, including without limitation tablets, capsules (solid- or liquid-filled), powders, granules, syrups and other liquids, etc.

For oral administration, any dose of ambrisentan that, together with the inhibitor or renin activity or release, is therapeutically effective, up to a maximum that is tolerated by the patient without unacceptable adverse side effects, can be administered. For most patients, such a dose is likely to be about 0.01 to about 50 mg/day, for example about 0.1 to about 25 mg/day or about 1 to about 10 mg/day. Higher or lower doses can be useful in specific circumstances.

The inhibitor of renin activity or release may also be present in the combination or composition, or used according to a method of the invention, in any dosage which, together with the ambrisentan, provides a beneficial effect without unacceptable adverse side-effects in the subject. Although, in one embodiment, the inhibitor of renin activity or release is orally bioavailable and is formulated for oral administration, the invention is not limited to any route of administration of the inhibitor of renin activity or release, so long as the route selected results in effective delivery of the drug to provide a beneficial effect. Further any suitable orally delivery dosage form may be used as indicated above for ambrisentan.

For oral administration, illustratively where the inhibitor of renin activity or release is aliskiren, a suitable dose is likely to be about 10 to about 1000 mg/day, for example about 30 to about 600 mg/day. Other renin inhibitors can be substituted at appropriate doses. One of skill in the art can readily identify a suitable dose for any particular renin inhibitor from publicly available information in printed or electronic form, for example on the internet.

The prescribed daily dosage amount of the ambrisentan and/or the inhibitor of renin activity or release can be administered in any suitable number of individual doses, for example four times, three times, twice or once a day. With a dosage form having appropriate controlled release properties, a lower frequency of administration may be possible, for example once every two days, once a week, etc. Administration can be continued for as long as clinically necessary, or for any desired duration, for example as prescribed by a physician. Thus duration of administration can illustratively be about one week to about one year or longer, and in some situations can be continued for substantially the remaining duration of the life of the subject.

Ambrisentan is suitable for once a day administration, and, where the inhibitor of renin activity or release is likewise suitable for once a day administration, it is generally most convenient to administer both the ambrisentan and the inhibitor of renin activity or release once a day at around the same time, for example, orally in the dosage amounts desired.

The ambrisentan and the inhibitor of renin activity or release may be formulated separately or together in a single pharmaceutical composition as discussed hereinbelow. Further, the ambrisentan and the inhibitor of renin activity or release may be administered by the same or different routes of administration, and at the same or different times such as those listed above. In one aspect, both the ambrisentan and the inhibitor of renin activity or release are each formulated for once-daily oral administration, in separate dosage forms or in a single composition.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, ambrisentan and the inhibitor of renin activity or release. In another example, the ambrisentan and the inhibitor of renin activity or release are separately packaged and available for sale independently of one another, but are co-marketed or co-promoted for use according to the invention. The separate dosage forms can also be presented to a subject separately and independently, for use according to the invention.

In another aspect of the invention, the combination can take the form of a pharmaceutical composition comprising the combination together with one or more pharmaceutically acceptable excipients. The composition can take any suitable form for the desired route of administration. Where the composition is administered orally, any suitable orally deliverable dosage form can be used, including without limitation tablets, capsules (solid- or liquid-filled), powders, granules, syrups and other liquids, etc.

Illustratively, a composition that is solid and orally deliverable, for example in a form of a tablet or capsule, typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function. Further information on excipients can be found in standard reference works such as Handbook of Pharmaceutical Excipients, 3rd ed. (Kibbe, ed. (2000), Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

In another embodiment of the invention, a method is provided for treating a hypertensive disorder in a subject. The method comprises administering to the patient in combination therapy ambrisentan and an inhibitor of renin activity or release in antihypertensive effective absolute and relative amounts, for example amounts as indicated hereinabove.

Examples of hypertensive disorders include not only pulmonary hypertension conditions, but also conditions marked by systolic hypertension, diastolic hypertension or both, including isolated systolic hypertension and hypertension in the elderly; such conditions can be primary (essential hypertension) or secondary to other conditions including obesity, diabetes, renal disorders (e.g., chronic renal failure, renovascular disease, diabetic nephropathy, etc.), adrenal disorders (e.g., adrenocortical and mineralocorticoid hypertension, pheochromocytoma, primary aldosteronism, Cushing's syndrome, etc.), insulin resistance, salt-sensitivity, polycystic ovary syndrome, sleep apnea, preeclampsia, thyroid and parathyroid diseases, and transplantation. Whether primary or secondary, such hypertension can be resistant to baseline antihypertensive therapies, including resistant hypertension as clinically defined or diagnosed.

In a particular aspect, the hypertensive disorder comprises PAH. PAH may be classified as either primary PAH, wherein vascular damage is present without any demonstrable cause, or secondary to another condition. Examples of such conditions include diseases of the scleroderma spectrum (e.g., mixed connective tissue disease, Raynaud's disease, CREST syndrome, systemic sclerosis, or overlap syndrome); rheumatoid arthritis; chronic hepatitis; systemic lupus erythematosus; anorexigen use; human immunodeficiency virus (HIV) infection; chronic hypoxemia resulting from conditions such as chronic bronchitis, emphysema, sleep apnea, interstitial lung disease, or pulmonary fibrosis; thromboembolic diseases such as in situ thrombosis, tumors, or sickle cell disease; volume and pressure overloads induced primarily from disorders of the left heart (for example, chronic heart failure, septal defects, mitral valve disease, and left atrial myxoma); and disorders directly affecting the pulmonary vasculature such as schistosomiasis, sarcoidosis and pulmonary capillary hemangiomatosis.

The subject can have mild, moderate or severe PAH. Subjects for whom the present method is especially useful have moderate to severe PAH. In particular aspects of the present invention, the subject exhibits initial pulmonary arterial pressure greater than about 25 mmHg, more especially greater than about 30 mmHg, and/or initial pulmonary vascular resistance greater than about 3 mmHg/L.min, more especially greater than about 5 mmHg/L.min.

In yet another embodiment of the invention, a method is provided for preventing one or more cardiovascular adverse events in a subject having a pulmonary hypertension condition, for example PAH. The method comprises administering to the subject in combination therapy ambrisentan and an inhibitor of renin activity or release in absolute and relative amounts effective to lower pulmonary arterial blood pressure, for example amounts as indicated hereinabove.

Examples of cardiovascular adverse effects include without limitation acute coronary syndrome (including unstable angina and non-Q wave infarction), myocardial infarction, heart failure, systolic heart failure, diastolic heart failure (also known as diastolic dysfunction), stroke, occlusive stroke, hemorrhagic stroke and combinations thereof. “Preventing” in the present context includes reducing risk, incidence and/or severity of a subsequent cardiovascular adverse effect.

In yet another embodiment of the invention, a method is provided for enhancing pulmonary function in a subject having a pulmonary hypertension condition, for example PAH. The method comprises administering to the patient in combination therapy ambrisentan and an inhibitor of renin activity or release in absolute and relative amounts effective to lower pulmonary arterial blood pressure, for example amounts as indicated hereinabove.

Pulmonary function can be evaluated in any of a number of ways. The present method can provide clinically meaningful improvements in at least one of exercise capacity, Borg dyspnea index immediately following exercise, WHO functional class and SF-36 health survey. Improvement can be measured by comparison with a baseline assessment prior to commencing a treatment regimen, and/or, in a clinical trial setting, by comparison with subjects receiving placebo.

Exercise capacity can be measured in any appropriate way known to one of skill in the art. A standard test for measuring exercise capacity is the 6-minute walk test, conducted for example according to American Thoracic Society (ATS) guidelines, or a modification thereof. In one aspect of the present embodiment, an improvement in pulmonary function following treatment as described herein is measurable by increase in distance walked in a 6-minute walk test. The increase can illustratively be at least about 10 m, for example at least about 20 m or at least about 30 m.

The Borg dyspnea index is a measure of breathlessness on a 0-10 scale, as rated by the subject, following exercise. Conveniently this measure is taken immediately after a 6-minute walk test. On this scale, 0 means no breathlessness and 10 means maximum breathlessness. In one aspect of the present embodiment, an improvement in pulmonary function following treatment as described herein is measurable by reduction of at least 1 index point, for example reduction of at least 2 index points.

WHO functional classes are defined as follows:
Class I: patients with pulmonary hypertension but without resulting limitation of physical activity; ordinary physical activity does not cause undue dyspnea or fatigue, chest pain, or near syncope;
Class II: patients with pulmonary hypertension resulting in slight limitation of physical activity but comfortable at rest; ordinary physical activity causes dyspnea or fatigue, chest pain, or near syncope;
Class III: patients with pulmonary hypertension resulting in marked limitation of physical activity but comfortable at rest; less than ordinary activity causes undue dyspnea or fatigue, chest pain, or near syncope;
Class IV: patients with pulmonary hypertension resulting in inability to carry out any physical activity without symptoms; signs of right heart failure manifested; dyspnea and/or fatigue may be present even at rest; discomfort increased by any physical activity.
   In one aspect of the present embodiment, an improvement in pulmonary function following treatment as described herein is sufficient to move a subject to at least one class above his or her baseline class, for example from Class III to Class II or from Class II to Class I.

The SF-36 (36-Item Short Form) health survey is a widely used instrument for assessing health and quality of life. See Ware & Sherbourne (1992) Med. Care 30(6):473-483. In one aspect of the present embodiment, an improvement in pulmonary function following treatment as described herein provides an improvement in quality of life as assessed by the SF-36 health survey.

In yet another embodiment of the invention, a method is provided for extending time to clinical worsening in a subject having PAH. The method comprises administering to the patient in combination therapy ambrisentan and an inhibitor of renin activity or release in absolute and relative amounts effective to lower pulmonary arterial blood pressure, for example amounts as indicated hereinabove.

“Clinical worsening” herein includes any one or more of the following events: hospitalization for PAH, atrial septostomy, lung transplantation or death. Time to clinical worsening can be established, for example, by records of subjects receiving treatment as described herein in comparison with subjects not receiving treatment for PAH, for example, in a clinical trial setting, patients receiving placebo. Any increase in time to clinical worsening can be beneficial; illustratively time to clinical worsening can be extended by at least about 10 days, for example by at least about 30 days, or by at least about 60 days.

Variants and illustrative modalities of each of the methods described herein, for example suitable inhibitors of renin activity or release, routes of administration, dosages, formulations, frequency and duration of administration for ambrisentan and the inhibitor of renin activity or release and suitable excipients are as described hereinabove for therapeutic combinations and pharmaceutical compositions of the invention. Thus any combination or composition embraced by the above description may be found suitable for use according to the present methods.

All patents and publications cited herein are incorporated by reference into this application in their entirety.

The words “comprise”, “comprises”, and “comprising” are to be interpreted inclusively rather than exclusively.

What is claimed is:

1. A therapeutic combination comprising ambrisentan and an inhibitor of renin activity or release.

2. The combination of claim 1, wherein the ambrisentan and the inhibitor of renin activity or release are present in antihypertensive effective absolute and relative amounts.

3. The combination of claim 1, wherein the ambrisentan and the inhibitor of renin activity or release are present in absolute and relative amounts effective to lower pulmonary arterial blood pressure in a patient having pulmonary arterial hypertension.

4. The combination of claim 3, wherein said absolute and relative amounts are effective to lower pulmonary arterial blood pressure by at least about 3 mmHg.

5. The combination of claim 1, wherein the inhibitor of renin activity or release is a renin inhibitor selected from the group consisting of aliskiren, ciprokiren, ditekiren, enalldren, remikiren, terlakiren, zankiren, and salts, esters, prodrugs, metabolites, enantiomers, racemates and tautomers thereof, and combinations thereof.

6. The combination of claim 1, wherein the ambrisentan and the inhibitor of renin activity or release are separately formulated for administration by the same or different routes at the same or different times.

7. The combination of claim 6, wherein at least the ambrisentan is formulated for oral administration.

8. The combination of claim 7, comprising ambrisentan in an amount providing a dose of about 0.1 to about 25 mg/day.

9. The combination of claim 7, comprising ambrisentan in an amount providing a dose of about 1 to about 10 mg/day.

10. The combination of claim 7, wherein the ambrisentan is formulated for once-daily oral administration.

11. The combination of claim 6, wherein the inhibitor of renin activity or release is orally bioavailable and is formulated for oral administration.

12. The combination of claim 11, wherein the inhibitor of renin activity or release comprises aliskiren in an amount providing a dose of about 10 to about 1000 mg/day.

13. The combination of claim 6, wherein the ambrisentan and the inhibitor of renin activity or release are each formulated for once-daily oral administration.

14. The combination of claim 1, in a form of a pharmaceutical composition comprising ambrisentan, the inhibitor of renin activity or release, and one or more pharmaceutically acceptable excipients.

15. The composition of claim 14, wherein the inhibitor of renin activity or release is orally bioavailable and the composition is formulated for oral administration.

16. The composition of claim 15, comprising ambrisentan in an amount providing a dose of about 0.1 to about 25 mg/day.

17. The composition of claim 15, comprising ambrisentan in an amount providing a dose of about 1 to about 10 mg/day.

18. The composition of claim 15, wherein the inhibitor of renin activity or release comprises aliskiren in an amount providing a dose of about 10 to about 1000 mg/day.

19. The composition of claim 15, that is formulated for once-daily oral administration.

20. The composition of claim 15, wherein the excipient(s) comprise one or more materials independently selected from the group consisting of diluents, binding agents, disintegrants, wetting agents and antifrictional agents.

21. A method for treating a hypertensive disorder in a subject, the method comprising administering to the subject in combination therapy ambrisentan and an inhibitor of renin activity or release in antihypertensive effective absolute and relative amounts.

22. The method of claim 21, wherein the hypertensive disorder is selected from the group consisting of pulmonary hypertension, essential hypertension, systolic hypertension, diastolic hypertension, isolated systolic hypertension, hypertension in the elderly, resistant hypertension and hypertension secondary to other conditions.

23. The method of claim 21, wherein the hypertensive disorder comprises pulmonary arterial hypertension.

24. The method of claim 23, wherein the pulmonary arterial hypertension is primary.

25. The method of claim 23, wherein the pulmonary arterial hypertension is secondary to another condition.

26. The method of claim 25, wherein the condition to which pulmonary arterial hypertension is secondary is selected from the group consisting of scleroderma, connective tissue disease, rheumatoid arthritis, chronic hepatitis, systemic lupus erythematosus, anorexigen use, HIV infection, chronic hypoxemia, thromboembolic diseases, volume and pressure overloads induced primarily from disorders of the left heart, schistosomiasis, sarcoidosis and pulmonary capillary hemangiomatosis.

27. The method of claim 23, wherein the subject exhibits initial pulmonary arterial pressure greater than about 25 mmHg.

28. The method of claim 23, wherein the subject exhibits initial pulmonary arterial pressure greater than about 30 mmHg.

29. The method of claim 23, wherein the subject exhibits initial pulmonary vascular resistance greater than about 3 mmHg/L/min.

30. The method of claim 23, wherein the subject exhibits initial pulmonary vascular resistance greater than about 5 mmHg/L/min.

31. The method of claim 21, wherein the inhibitor of renin activity or release is a renin inhibitor selected from the group consisting of aliskiren, ciprokiren, ditekiren, enalkiren, remikiren, terlakiren, zankiren, and salts, esters, prodrugs, metabolites, enantiomers, racemates and tautomers thereof, and combinations thereof.

32. The method of claim 21, wherein the ambrisentan and the inhibitor of renin activity or release are administered as separate formulations by the same or different routes at the same or different times.

33. The method of claim 21, wherein the ambrisentan and the inhibitor of renin activity or release are administered together in a single pharmaceutical composition.

34. The method of claim 21, wherein at least the ambrisentan is administered orally.

35. The method of claim 34, wherein the ambrisentan is administered at a dose of about 0.1 to about 25 mg/day.

36. The method of claim 34, wherein the ambrisentan is administered at a dose of about 1 to about 10 mg/day.

37. The method of claim 34, wherein the ambrisentan is orally administered once daily.

38. The method of claim 34, wherein the inhibitor of renin activity or release is orally bioavailable and is administered orally.

39. The method of claim 38, wherein the inhibitor of renin activity or release comprises aliskiren in an amount providing a dose of about 10 to about 1000 mg/day.

40. The method of claim 38, wherein the ambrisentan and the inhibitor of renin activity or release are each administered orally once daily.

41. The method of claim 40, wherein the ambrisentan and the inhibitor of renin activity or release are administered together in a single pharmaceutical composition.

42. The method of claim 41, wherein the composition further comprises one or more excipients independently selected from the group consisting of diluents, binding agents, disintegrants, wetting agents and antifrictional agents.

43. A method for preventing one or more cardiovascular adverse events in a subject having a pulmonary hypertension condition, the method comprising administering to the subject in combination therapy ambrisentan and an inhibitor of renin activity or release in absolute and relative amounts effective to lower pulmonary arterial pressure.

44. The method of claim 43, wherein the one or more cardiovascular adverse events are selected from the group consisting of acute coronary syndrome, myocardial infarction, heart failure, systolic heart failure, diastolic heart failure, stroke, occlusive stroke, hemorrhagic stroke and combinations thereof.

45. A method for enhancing pulmonary function in a subject having a pulmonary hypertension condition, the method comprising administering to the subject in combination therapy ambrisentan and an inhibitor of renin activity or release in absolute and relative amounts effective to lower pulmonary arterial pressure.

46. The method of claim 45, wherein enhanced pulmonary function is demonstrated by improvement in at least one of exercise capacity, Borg dyspnea index, WHO functional class and SF-36 health survey.

47. The method of claim 45, wherein enhanced pulmonary function is demonstrated by improvement in exercise capacity as measured by an increase of at least about 10 m in distance walked in a standard 6-minute walk test.

48. A method for extending time to clinical worsening in a subject having a pulmonary hypertension condition, the method comprising administering to the subject in combination therapy ambrisentan and an inhibitor of renin activity or release in absolute and relative amounts effective to lower pulmonary arterial pressure.

49. The method of claim 48, wherein the time to clinical worsening is extended by at least about 10 days.
